(19) United States
(12) Patent Application Publication
(10) Pub. No.: US 2025/0073157 A1
(43) Pub. Date: Mar. 6, 2025

(54) COMPOSITIONS AND METHODS FOR TREATING AND PREVENTING HAIR LOSS

(71) Applicant: Vias Partners, LLC, Doylestown, PA (US)

(72) Inventor: Sanjay BATRA, New Hope, PA (US)

(21) Appl. No.: 18/726,390

(22) PCT Filed: Jan. 4, 2023
(86) PCT No.: PCT/US2023/010146

§ 371 Date: Jul. 2, 2024

Related U.S. Application Data
(60) Provisional application No. 63/296,436, filed on Jan. 4, 2022.

(51) Int. Cl. A61K 8/97; A61Q 7/00

(52) U.S. Cl. CPC A61K 8/97; A61Q 7/00; A61K 2800/5922; A61K 2800/87

(57) ABSTRACT

Described herein are compositions for promoting hair growth and/or treating or preventing hair loss in a mammalian subject. In certain embodiments, the mammalian subject is a human male or a human female. Methods of making and using the same are also described. The compositions in various embodiments may comprise: a tea extract which may comprise a polyphenol, e.g., epigallocatechin gallate; an extract from Larix europaea which may comprise the flavonoid taxifolin; a natural ingredient that antagonizes or mitigates the proliferation of Gram-positive bacteria in the hair shaft passageway; compounds including zinc chloride and/or hexamidine diisethionate; follicle stimulator. e.g., caffeine, an ingredient that improves the health of the dermis. e.g., sodium hyaluronate; and/or ingredients specifically tailored for male or female subjects, e.g., Serenoa serrulata fruit extract or extract of Pterocarpus marsupium.

### CROSS-REFERENCE TO RELATED APPLICATION

This application claims the benefit of priority of U.S. Provisional Patent Application No. 63/296,436, entitled “Compositions and Methods for Treating and Preventing Hair Loss,” filed Jan. 4, 2022, the entire disclosure of which is hereby incorporated herein by reference.

### BACKGROUND

Androgenetic alopecia (AGA) is the most common cause of hair loss that is characterized by non-scarring, progressive loss of terminal hairs. In men, hair loss typically involves the temporal and vertex regions of the scalp, while female patterned hair loss is more diffuse and typically around the frontal and vertex regions. AGA features progressive follicular miniaturization that results in the vellus transformation of terminal hairs within areas of hair loss. Additionally, an alteration of the hair cycle occurs in AGA as the anagen phase, which is primarily the active hair growth phase, shortens.

Patients suffering from AGA report psychosocial effects attributed to the condition as hair loss may negatively affect feelings of attractiveness, self-esteem, and body image. Although AGA causes a significant impact on the quality of life of patients, the approved amount of therapeutic options are limited and patients may not respond to first-line therapies. Only two medications, oral finasteride and topical minoxidil, have been FDA-approved for the treatment of AGA. Nutraceuticals, a class of naturally derived therapeutics from botanicals, have become an emerging option for hair loss in patients who have limited efficacy and suffer side effects. The use of botanicals is safe and has activity against the causative components of hair loss, such as inflammation, oxidative damage, stress mediators, and dihydrotestosterone. The use of natural ingredients are often used in combination therapy to treat AGA.

However, there remains a need for topical products that are easy to use and do not result in the side effects associated with current offerings. Certain embodiments of the present invention are designed to meet these, and other, ends.

### SUMMARY

In some embodiments, the present invention provides a composition for use in treating or preventing hair loss in a mammalian subject. In other embodiments, the compositions described herein are delivered through a dispenser comprising a roller ball. In certain embodiments, the mammalian subject is a human male. In some embodiments, the mammalian subject is a human female.

In some embodiments, the compositions of the present invention comprise a tea extract (Camelia sinensis). In some embodiments, the tea extract comprises a polyphenol. In some embodiments, the polyphenol comprises epigallocatechin gallate (EGCG). In some embodiments, the compositions of the present invention comprise an extract from horse chestnut (Aesculus hippocastanum). In some embodiments, the compositions of the present invention improve the vasculature and facilitate the removal of toxins from the scalp.

In further embodiments, the present invention provides compositions comprising an extract from the wood of European Larch (Larix europaea). In some embodiments, the extract from the wood of European Larch is rich in the flavonoid taxifolin, a potent antioxidant. This is important as the conversion of sugars to fats in AGA generates toxins, which are also known as reactive oxygen species or ‘free radicals.’ In some embodiments, the taxifolin complements the effects of horse chestnut by increasing the anti-inflammatory effect of the formula.

Some embodiments of the present invention provide compositions comprising a natural ingredient that antagonizes or mitigates the proliferation of Gram-positive bacteria. In some embodiments, the ingredient that antagonizes or mitigates the proliferation of Gram-positive bacteria reduces microbial overgrowth in the hair shaft passageway. In some embodiments, the Gram-negative bacterium is Propionibacterium acnes. This bacterium works in collaboration with the fungus that causes dandruff in some people, called Malassezia furfur, which is another oil loving microbe. The orchestration of inflammation and oil production by these two microbes is defeated by using antimicrobial therapies. In some embodiments, the ingredient that antagonizes or mitigates the proliferation of Gram-positive bacteria comprises a prenylated isoflavone. In some embodiments, the prenylated isoflavone is derived from licorice (Glycyrrhiza uralensis).

In certain embodiments, the compositions of the present invention comprise a zinc compound. In some embodiments, the zinc compound comprises zinc chloride. Still further embodiments provide compositions comprising hexamidine diisethionate.

In other embodiments, the compositions of the present invention comprise a follicle stimulator. In some embodiments, the follicle stimulator comprises caffeine, or a derivative thereof. Further embodiments provide compositions comprising a mixture of tripeptides. In some embodiments, the mixture of tripeptides comprises peptides having a unique penetrating ability, so each unique peptide reaches a different type of tissue and does its work there.

In some embodiments, compositions of the present invention comprise an ingredient that improves the health of the dermis (e.g., sodium hyaluronate, which is a building block of the extracellular matrix). Still further embodiments provide compositions comprising a penetration enhancer. In some embodiments, the penetration enhancer comprises menthol and Mentha piperita oil.

In some embodiments, the composition is specifically tailored for male or female mammalian subjects. In some embodiments, the male formula comprises saw palmetto (Serenoa serrulata) fruit extract. In some embodiments, the female formula comprises isoflavones, sorbitol, glutamic acid and extract of Pterocarpus marsupium.

Some embodiments of the present invention provide compositions having a viscosity that ensures delivery through an applicator comprising a rollerball. In some embodiments, the rollerball has a compressibility that ensures adequate delivery of the compositions described herein to a skin surface (e.g., the scalp surface). In some embodiments, the compositions of the present invention have a viscosity that ensures delivery of an effective amount of active ingredient to hair follicle stem cells, stem cell bulbs, the base of the hair follicle and/or bulge stem cells. Other embodiments of the present invention provide compositions having a viscosity that ensures transdermal delivery/penetration across the scalp surface. In some embodiments, the compositions of the present invention may provide a yellowish hue to the scalp surface and/or hair follicle.

Other embodiments of the present invention provide a hair care composition comprising: an androgen balancing agent (e.g., a 5α-reductase inhibitor/a dihydrotestosterone (DHT) blocker); a prostaglandin balancing agent; an antimicrobial agent; an anti-inflammatory agent; a collagen modulator; a circulation enhancer; an amino acid; and a cosmetically acceptable carrier.

Further embodiments of the present invention provide a composition for promoting hair growth and/or reducing hair loss comprising: a copper ion source; an extract or derivative of Camellia sinensis; an extract or derivative of Serenoa serrulate; and a natural vasodilator.

The compositions and methods of the present invention may be applied to patients suffering from hair loss or in healthy patients simply wanting to increase hair growth in any part of the body.

The compositions disclosed herein are formulated for topical administration. The term “topical administration” as used herein includes applying a formulation as described herein to the outer skin or hair. The application will generally occur at or near the area of desired hair growth.

Accordingly, appropriate formulation or composition types include, without limitation, solutions, gels, ointments, foams, films, liniments, creams, shampoos, lotions, pastes, jellies, sprays and aerosols. Such formulation types can be applied in swaths, patches, applicators or through the use of impregnated dressings depending on the situation and part of the body to be treated.

Typically, the formulations described herein will be applied repeatedly for a sustained period of time to the part of the body to be treated. In particular embodiments, formulations disclosed herein can include one or more applications daily, one or more applications weekly, one or more applications monthly or one or more applications yearly for a period of treatment of at least one day, at least one week, at least one month, at least one year or until the treatment has achieved or achieved and maintained a desired result.

Formulations described herein will be administered in safe and effective amounts. As used herein, “safe and effective amounts” include an amount sufficient so that the composition provides the desired hair growth stimulation effect at a reasonable benefit/risk ratio attendant with any medical treatment. Within the scope of sound medical judgment, the amount of active components used can vary with the particular condition being treated, the severity of the condition, the cause of the condition, the duration of the treatment, the specific active component employed, its concentration, the specific vehicle utilized, the general health of the patient, the tolerance of the patient to various effects of the administration, other drugs being administered to the patient, and like factors within the specific knowledge and expertise of the patient or attending physician.

### BRIEF DESCRIPTION OF THE DRAWINGS

FIG. 1 depicts a male scalp vertex at baseline, at 4 months and at 8 months after treatment with an exemplary composition of the present invention.

FIG. 2 depicts a male scalp close-up at baseline and at 4 months after treatment with an exemplary composition of the present invention.

FIG. 3 depicts another male scalp vertex at baseline and at 4 months after treatment with an exemplary composition of the present invention.

FIG. 4 depicts another male scalp vertex at baseline and at 4 months after treatment with an exemplary composition of the present invention.

FIG. 5 depicts a female frontal scalp at baseline and at 4 months after treatment with an exemplary composition of the present invention.

FIG. 6 depicts another male scalp vertex at baseline and after 6 weeks of treatment with an exemplary composition of the present invention.

FIG. 7A and FIG. 7B depict a second male vertex scalp at baseline and after 6 weeks, respectively, of treatment with an exemplary composition of the present invention.

FIG. 8 depicts another male vertex scalp at baseline and after 6 weeks of treatment with an exemplary composition of the present invention.

FIG. 9 depicts another male vertex scalp at baseline and after 6 weeks of treatment with an exemplary composition of the present invention.

FIG. 10 depicts a female vertex scalp at baseline (upper two panels) and after 6 weeks (lower two panels) of treatment with an exemplary composition of the present invention.

FIG. 11 depicts another female vertex scalp at baseline (upper two panels) and after 6 weeks (lower two panels) of treatment with an exemplary composition of the present invention.

FIG. 12 is a graph illustrating the difference in hair thickness and growth between baseline and after 8 weeks of treatment with an exemplary composition of the present invention.

FIG. 13A and FIG. 13B depict a comparison for a male and a female patient, respectively, after completion of the study described in Example 2 (below).

FIG. 14 depicts the results of a patient satisfaction questionnaire after 8 weeks of treatment with an exemplary composition of the present invention, in accordance with the study described in Example 2 (below).

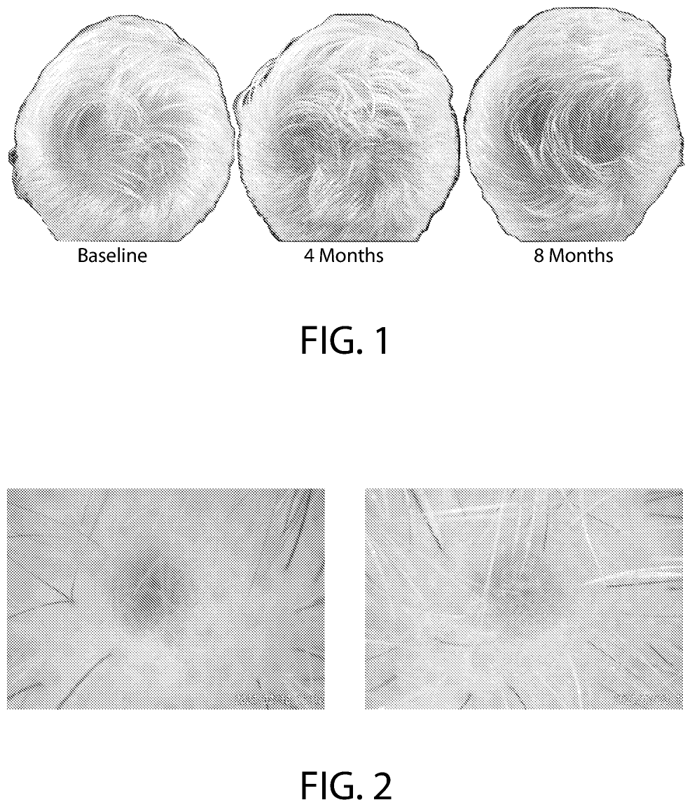

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment shown or described. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2.0 wt. %” refers to a number between and including 1.800 wt. % and 2.200 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the hair care composition. The symbol “°” refers to a degree, such as a temperature degree or a degree of an angle. The symbols “h”, “min”, “mL”,” nm”, “μm” means hour, minute, milliliter, nanometer, and micrometer, respectively. The abbreviation “UV-VIS” as referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond respectively.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the hair care compositions of the instant disclosure can be free or essentially free of all components and elements positively recited throughout the instant disclosure. In some instances, the hair care compositions of the present disclosure may be substantially free of non-incidental amounts of the ingredient(s) or compound(s) described herein. A non-incidental amount of an ingredient or compound is the amount of that ingredient or compound that is added into the hair care composition by itself. For example, a hair care composition may be substantially free of a non-incidental amount of an ingredient or compound, although such ingredient(s) or compound(s) may be present as part of a raw material that is included as a blend of two or more compounds.

As used herein, “topical application” means directly laying on or spreading on outer skin using, e.g., by use of the hands or an applicator such as a wipe, puff, roller, or spray.

As used herein, “cosmetically-acceptable” means that the product(s) or compound(s) which the term describes are suitable for use in contact with tissues (e.g., the skin) without undue toxicity, incompatibility, instability, irritation, allergic response, and the like. This term is not intended to limit the ingredient/product, which it describes to use solely as a cosmetic (e.g., the ingredient/product may be used as a pharmaceutical).

As used herein, “topical carrier” means one or more compatible solid or liquid filler diluents that are suitable for topical administration to a mammal. Examples of topical carriers include, but are not limited to, water, waxes, oils, emollients, emulsifiers, thickening agents, gelling agents, and mixtures thereof.

As used herein, “hair” means scalp, head, facial and/or body hair, including but not limited to the scalp, eye lashes, brows, mustache, beard, ear, nasal, chest, pubic, auxiliary and the like.

As used herein, “hair care composition” is intended to include, but is not limited to, a composition that induces/promotes hair growth and/or reduces hair loss.

As used herein, “inducing hair growth” means the earlier induction of growth of a new hair cycle, and/or prolonging the active growth phase (anagen) of the hair cycle and/or increasing the growth rate of the hair and/or increasing the width of hair shaft, including, but not limited to, the induction of the growth of hair and making it more visible to the eye.

As used herein, “improving hair quality” means increasing the diameter of the hair shaft and/or enhancing the visual attributes of the hair like hair volume, hair shine and hair thickness, and/or affecting the characteristics of the hair shaft and/or hair cuticles, including, but not limited to, creating a smoother look or feel, and/or increase in shine.

In some embodiments, the present invention provides a hair care composition comprising: an androgen balancing agent (e.g., a 5α-reductase inhibitor/a dihydrotestosterone (DHT) blocker); a prostaglandin balancing agent; an antimicrobial agent; an anti-inflammatory agent; a collagen modulator; a circulation enhancer; an amino acid; and a cosmetically acceptable carrier.

In some embodiments, the androgen balancing agent is selected from an extract or derivative of: Ganoderma lucidum (Reishi); Glycyrrhiza glabra (Licorice); Paeonia lactiflora (White Peony); Camellia sinensis (Green Tea); Mentha spicata (Spearmint); Actaea racemose (Black Cohosh); Vitex agnus-castus (Chaste Tree); Serenoa repens (Saw Palmetto); and a combination of two or more thereof.

In certain embodiments, the antimicrobial comprises an antibacterial agent or an antifungal agent. In other embodiments, the antimicrobial agent is selected from: a zinc ion source (e.g., zinc chloride or zinc gluconate); Glycyrrhiza uralensis (Licorice) root extract; hexamidine diisethionate; Saccharomyces/copper ferment; Saccharomyces/zinc ferment; ketonconazole; and a combination of two or more thereof.

Further embodiments of the present invention provide hair care compositions, wherein the anti-inflammatory agent is selected from: Larix europaea wood extract (e.g., taxifolin or DHQG); Aesculus hippocastanum (Horse Chestnut) seed extract; Phellinus linteus (mushroom) extract; apigenin; oleanolic acid; Aloe barbadensis leaf extract; Houttuynia cordata extract; and a combination of two or more thereof.

In some embodiments, the collagen modulator comprises hyaluronic acid, or a salt thereof.

Other embodiments of the present invention further comprise a penetration enhancer. In further embodiments, the penetration enhancer is selected from: peppermint oil; isopropyl myristate; a ceramide; lecithin; a liposome; and a combination of two or more thereof.

In certain embodiments, the hair care composition is a topical composition. In some embodiments, the hair care composition is selected from: a lotion; a serum; a tincture; a cream; an ointment; a gel; a paste; a powder; and a balm.

In some embodiments, the circulation enhancer comprises a vasodilator. In some embodiments, the vasodilator is selected from: caffeine; cocoa; coenzyme Q10 (CoQ10); a source of Allium sativum; arginine; magnesium; niacin (vitamin B3); a nitrate source; Petroselinum crispum; Zingiber officinale; and a combination of two or more thereof.

Further embodiments of the present invention further comprise a peptide selected from: copper-tripeptie-1; glycyl-L-histidyl-L-lysine; palmitoyl tripeptide-1; biotinoyl tripeptide-1; and a combination of two or more thereof.

In other embodiments, the amino acid is selected from: glutamic acid; methionine; cysteine; homocysteine; taurine; and a combination of two or more thereof.

In some embodiments, the hair care composition further comprises soy isoflavones.

In some embodiments, the hair care composition further comprises a sugar alcohol. In certain embodiments, the sugar alcohol is selected from: mannitol; xylitol; sorbitol; and a combination of two or more thereof.

In some embodiments, the cosmetically acceptable carrier comprises: from about 50 wt. % to about 80 wt. % water; from about 5 wt. % to about 20 wt. % of a monohydric alcohol; and from about 1 wt. % to about 15 wt. % of a diol. In other embodiments, the cosmetically acceptable carrier comprises: from about 55 wt. % to about 70 wt. % water; from about 7 wt. % to about 15 wt. % of a monohydric alcohol; and from about 5 wt. % to about 10 wt. % of a diol. In some embodiments, the monohydric alcohol comprises ethanol. In some embodiments, the diol is selected from propylene glycol; butylene glycol; and a combination thereof.

Other embodiments of the present invention provide a composition for promoting hair growth and/or reducing hair loss comprising: a copper ion source; an extract or derivative of Camellia sinensis; an extract or derivative of Serenoa serrulate; and a natural vasodilator. In some embodiments, the natural vasodilator comprises a methylxanthine (e.g., caffeine).

Further embodiments of the present invention provide a method for promoting hair growth and reducing hair loss comprising: administering an effective amount of a hair care composition as described herein to a skin surface of a subject in need thereof.

In some embodiments, the present invention provides compositions and methods for alleviating hair loss; and may be effective to improve hair loss caused by, for example, forms of alopecia including androgenetic alopecia, alopecia areata, alopecia mucinosa, telogen effluvium, chronic inflammation, extreme and/or chronic stress, diabetes, cellulitis, hair treatments, hereditary disorders, hormonal changes, hyperthyroidism, hypothyroidism, malnutrition, lupus, medication side effects, including from chemotherapy and birth control, radiation, aging and trichotillomania.

Some embodiments of the present invention provide methods for initiating aggregation of intradermal adipocytes on the scalp of a mammalian subject, comprising: administering any one of the hair care compositions described herein to a skin surface of a mammalian subject. Further embodiments of the present invention provide methods for aggregating intradermal adipocytes on the scalp of a mammalian subject, comprising: administering any one of the hair care compositions described herein to a skin surface of a mammalian subject. Still further embodiments of the present invention provide methods for increasing aggregation of intradermal adipocytes on the scalp of a mammalian subject, comprising: administering any one of the hair care compositions described herein to a skin surface of a mammalian subject. In some embodiments, the aggregation of intradermal adipocytes results in a yellowing of the scalp.

In some embodiments, the compositions of the present invention contain only natural ingredients. Accordingly, in certain embodiments, compositions of the present invention may be administered from about once/day (QD) to about every 30 minutes (Q 30 mins). In some embodiments, the compositions of the present invention may be administered from about twice/day (BID) to about every 60 minutes. In other embodiments, the compositions of the present invention may be administered from about three times/day (TID) to about once every 2 hours. In yet other embodiments, the compositions of the present invention may be administered from about 4 times/day (QID) to about once every 4 hours. In some embodiments, the compositions of the present invention are administered twice a day.

In some embodiments, the compositions of the present invention are administered for at least about 1 week. In some embodiments, the compositions of the present invention are administered for at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 10 years, at least about 15 years, at least about 20 years, at least about 25 years, at least about 30 years, at least about 35 years, at least about 40 years, or at least about 50 years.

In some embodiments, the compositions of the present invention are administered for at about 1 week. In some embodiments, the compositions of the present invention are administered for about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 3 months, about 4 months, about 5 months, about 6 months, about 7 months, about 8 months, about 9 months, about 10 months, about 11 months, about 1 year, about 2 years, about 3 years, about 4 years, about 5 years, about 10 years, about 15 years, about 20 years, about 25 years, about 30 years, about 35 years, about 40 years, or about 50 years.

In some embodiments, the performance/efficacy of the compositions and methods of the present invention are evaluated according to the GRO Index. The GRO Index is a method to track hair growth and hair loss (see, e.g., https://www.grotrack.cloud). Specifically, the GRO Index quantifies-inter alia-hair mass density, which is used to track hair growth from both global images and macro images. Hair growth is presented as a percentage change from a subject's baseline assessment.

In some embodiments, the GRO Index is calculated after about 1 week of treatment with a composition of the present invention. In some embodiments, the GRO Index is calculated after about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 3 months, about 4 months, about 5 months, about 6 months, about 7 months, about 8 months, about 9 months, about 10 months, about 11 months, about 1 year, about 2 years, about 3 years, about 4 years, about 5 years, about 10 years, about 15 years, about 20 years, about 25 years, about 30 years, about 35 years, about 40 years, or after about 50 years of treatment with a composition of the present invention.

In some embodiments, the compositions of the present invention increase the GRO Index by at least about 5, after about 6 weeks of treatment in accordance with any one of the methods described herein. In other embodiments, the compositions of the present invention increase the GRO Index by from about 5 to about 50, after about 6 weeks of treatment in accordance with any one of the methods described herein. In some embodiments, the compositions of the present invention increase the GRO Index by at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, after about 6 weeks of treatment in accordance with any one of the methods described herein In some embodiments, the compositions of the present invention increase the GRO Index by about 10, about 11, about 12, about 13, about 14, about 15, about 16, about 17, about 18, about 19, or about 20, after about 6 weeks of treatment in accordance with any one of the methods described herein.

In some embodiments, the topical hair care compositions of the present invention are co-administered with an oral composition. In some embodiments, the oral composition comprises an extract of Curcuma longa, an extract of Withania somnifera, and an extract of Serenoa serrulata, to said subject.

In some embodiments, the oral composition comprises from about 100 to about 1000 mg of an extract of Curcuma longa, from about 100 mg to about 1000 mg of an extract of the roots and leaves of Withania somnifera, and at least about 200 mg of saw palmetto extract. In other embodiments, the oral composition further comprises from about 100 mg to about 1000 mg of tocotrienols and/or tocopherols.

In further embodiments, the oral composition may further comprise low molecular weight collagen and hyaluronic acid and the collagen may be Type I and Type III collagen and have a molecular weight of less than about 5000 daltons. Still further embodiments provide oral compositions wherein the collagen may be present in an amount from about 500 mg to about 2000 mg, and said hyaluronic acid may be present in an amount from about 100 mg to about 1000 mg.

In certain embodiments, the oral compositions of the present invention may include at least about 200 mg of curcumin, at least about 100 mg of withanolides, at least about 200 mg of saw palmetto extract, and at least about 100 mg of tocotrienols and/or tocopherols comprising a complex of 78% tocotrienols and 22% tocopherols, at least about 1000 mg of Type I and III collagen having a molecular weight of less than about 3000 Daltons, and at least about 100 mg of hyaluronic acid.

In some embodiments, the oral compositions of the present invention may further include piperine. In some embodiments, the oral compositions of the present invention may further include at least about 5 mg of piperine.

In other embodiments, the oral compositions of the present invention may further include one or more compounds from the group consisting of green tea extract, L-methionine, L-cysteine hydrochloride, resveratrol, capsaicin, biotin, selenium, Vitamin D, Vitamin A, and Vitamin C.

In some embodiments, the hair care composition is administered from a dispenser. In some embodiments, the dispenser comprises an applicator. In some embodiments, the applicator comprises a roller ball. In other embodiments, the applicator comprises a perforated surface. In some embodiments, the dispenser comprises a reservoir. In some embodiments, the reservoir is fluidly coupled to the applicator. In some embodiments, the hair care composition is maintained within the reservoir. In some embodiments, the hair care composition has a viscosity adapted to be delivered through the perforated surface of said applicator.

In some embodiments, the perforated surface has from about 1 to about 100 apertures/mm². In some embodiments, the perforated surface has from about 1 to about 90 apertures/mm². In some embodiments, the perforated surface has from about 1 to about 80 apertures/mm². In some embodiments, the perforated surface has from about 1 to about 75 apertures/mm². In some embodiments, the perforated surface has from about 1 to about 70 apertures/mm². In some embodiments, the perforated surface has from about 1 to about 65 apertures/mm². In some embodiments, the perforated surface has from about 1 to about 60 apertures/mm². In some embodiments, the perforated surface has from about 1 to about 55 apertures/mm². In some embodiments, the perforated surface has from about 1 to about 50 apertures/mm². In some embodiments, the perforated surface has from about 1 to about 45 apertures/mm². In some embodiments, the perforated surface has from about 1 to about 40 apertures/mm². In some embodiments, the perforated surface has from about 1 to about 35 apertures/mm². In some embodiments, the perforated surface has from about 1 to about 30 apertures/mm². In some embodiments, the perforated surface has from about 1 to about 25 apertures/mm². In some embodiments, the perforated surface has from about 1 to about 20 apertures/mm².

In some embodiments, the hair care composition may comprise Aralia Quinquetolia, Astragalus Glycyphyllos, Angelica Arhangelica Root, Salvia Officinalis, Capsicum, Carya Alba, Corthamis Tinctorius, Cortex dictamni radicis, Flos Chrysanthemum, Heshouwu, Iron-Fist Ginseng, Miltiorrhizae, Notoginseng, Paorulca Glandulosa, Peach Kernel Oil, Rhizome of Szechuan Lovage, Radix astragali, Radix Ginseng, Radix Polygoni Multiflori, Red-rooted Salvia, Rhizhoma gastroidia ginseng, Seu radix notopterygii, and/or Sophera flavescens.

Some embodiments of the present invention provide hair care compositions that comprise a vitamin. In some embodiments, the vitamin suitable for use in the hair care compositions of the present invention may include, but is not limited to, vitamin A, vitamin Bs such as vitamin B3, vitamin B5, and vitamin B12, vitamin C, vitamin K, vitamin E such as alpha, gamma or delta-tocopherol, and derivatives (such as salts and esters) and mixtures thereof.

In some embodiments, the hair care compositions of the present invention contain an antioxidant. Examples of antioxidants which may be utilized in the compositions and methods of this invention include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), different types of tocopherols (e.g., alpha-, gamma-, and delta-tocopherols and their esters such as acetate) and their mixtures, tocotrienols, and ubiquinone. Natural extracts containing antioxidants suitable for use in the compositions of this invention, include, but not limited to, extracts containing flavonoids, isoflavonoids, and their derivatives such as genistein and daidzein (e.g., such as soy and clover extracts, extracts containing resveratrol and the like. Examples of such natural extracts include grape seed, green tea, pine bark, and propolis.

In some embodiments, the composition may include one or more humectant(s). The one or more humectant(s) may be present in an amount from about 0.5 to about 30 wt. %, based on the total weight of the hair care composition. For example, the hair care composition may include humectant(s) in an amount from about 0.5 to about 30 wt. %, about 0.5 to about 28 wt. %, about 0.5 to about 26 wt. %, about 0.5 to about 24 wt. %, about 0.5 to about 22 wt. %, about 0.5 to about 20 wt. %, about 0.5 to about 16 wt. %, about 0.5 to about 14 wt. %, about 0.5 to about 12 wt. %, about 0.5 to about 10 wt. %, about 0.5 to about 8 wt. %, about 0.5 to about 6 wt. %, about 0.5 to about 4 wt. %, about 0.5 to about 2 wt. %; from about 1 to about 28 wt. %, about 1 to about 26 wt. %, about 1 to about 24 wt. %, about 1 to about 22 wt. %, about 1 to about 20 wt. %, about 1 to about 16 wt. %, about 1 to about 14 wt. %, about 1 to about 12 wt. %, about 1 to about 10 wt. %, about 1 to about 8 wt. %, about 1 to about 6 wt. %, about 1 to about 4 wt. %; from about 2 to about 28 wt. %, about 2 to about 26 wt. %, about 2 to about 24 wt. %, about 2 to about 22 wt. %, about 2 to about 20 wt. %, about 2 to about 16 wt. %, about 2 to about 14 wt. %, about 2 to about 12 wt. %, about 2 to about 10 wt. %, about 2 to about 8 wt. %, about 2 to about 6 wt. %; from about 3 to about 30 wt. %, about 3 to about 28 wt. %, about 3 to about 26 wt. %, about 3 to about 24 wt. %, about 3 to about 22 wt. %, about 3 to about 20 wt. %, about 3 to about 16 wt. %, about 3 to about 14 wt. %, about 3 to about 12 wt. %, about 3 to about 10 wt. %, about 3 to about 8 wt. %; from about 6 to about 30 wt. %, about 6 to about 28 wt. %, about 6 to about 26 wt. %, about 6 to about 24 wt. %, about 6 to about 22 wt. %, about 6 to about 20 wt. %, about 6 to about 16 wt. %, about 6 to about 14 wt. %, about 6 to about 12 wt. %; from about 9 to about 30 wt. %, about 9 to about 28 wt. %, about 9 to about 26 wt. %, about 9 to about 24 wt. %, about 9 to about 22 wt. %, about 9 to about 20 wt. %, about 9 to about 16 wt. %, or about 9 to about 14 wt. %, including all ranges and subranges thereof, based on the total weight of the hair care composition.

Examples of humectants that may be incorporated in certain embodiments include triethylene glycol, tripropylene glycol, propylene glycol, glycerin, polyethylene glycols, PPG, glycerin, sorbitol, hexylene glycol, butylene glycol, urea, collagen, aloe, honey, hyaluronic acid, 1,2 hexanediol, saccharide isomerate and a combination of two or more thereof. In some cases, the one or more humectants comprise saccharide isomerate and, optionally, one or more humectants.

Additional examples of humectants include certain polyols and/or glycols. The term “polyol” should be understood as meaning, within the meaning of the present disclosure, an organic molecule comprising at least two free hydroxyl groups. The polyols of the hair care composition may be glycols or compounds with numerous hydroxyl groups. In some cases, the one or more polyols is/are selected from the group consisting of C2-C32 polyols. The one or more polyols may have from 2 to 32 carbon atoms, from 3 to 16 carbon atoms, or from 3 to 12 carbon atoms. Non-limiting examples of polyols that may be included in the hair care composition, in certain instances, include ethylene glycol, propylene glycol, butylene glycol, hexylene glycol, diglycerin, diethylene glycol, and dipropylene glycol, and mixtures thereof.

The hair care composition may include one or more pH adjusters to increase or decrease the overall pH of the hair care composition. For example, one or more acids may be included to decrease the pH of the hair care composition. Examples of suitable acids for decreasing the pH of the hair care composition include, but are not limited to, citric acid, acetic acid, and the like. The hair care composition may include one or more bases, such as sodium hydroxide, potassium hydroxide and the like, to increase the pH of the hair care composition. Additional or alternative acids and bases that are suitable for adjusting the pH of the hair care composition are readily known to one of ordinary skill in the art.

The amount of the pH adjuster in the hair care composition may be based on the desired pH of the final hair care composition and/or product. For example, the total amount of the pH adjuster may range from about 0.05 to about 50 wt. %, based on the total weight of the hair care composition. In some instances, the total amount of pH adjuster is from about 0.05 to about 20 wt. %, about 0.05 to about 15 wt. %, about 0.1 to about 10 wt. %, or about 0.12 to about 5 wt. %, including ranges and sub-ranges therebetween, based on the total weight of the hair care composition.

In certain embodiments, the hair care composition has a pH of from about 2 to about 7. For example, the pH of the hair care composition may be from about 2 to about 7, about 2 to about 6.5, about 2 to about 6, about 2 to about 5.5, about 2 to about 5, about 2 to about 4.5, about 2 to about 4, about 2 to about 3.5; from about 2.5 to about 7, about 2.5 to about 6.5, about 2.5 to about 6, about 2.5 to about 5.5, about 2.5 to about 5, about 2.5 to about 4.5, about 2.5 to about 4, about 2.5 to about 3.5; from about 3 to about 7, about 3 to about 6.5, about 3 to about 6, about 3 to about 5.5, about 3 to about 5, about 3 to about 4.5, about 3 to about 4; from about 3.5 to about 7, about 3.5 to about 6.5, about 3.5 to about 6, about 3.5 to about 5.5, about 3.5 to about 5, about 3.5 to about 4.5; from about 4 to about 7, about 4 to about 6.5, about 4 to about 6, about 4 to about 5.5, about 4 to about 5; from about 4.5 to about 7, about 4.5 to about 6.5, about 4.5 to about 6, about 4.5 to about 5.5; from about 5 to about 7, about 5 to about 6.5, about 5 to about 6; from about 5.5 to about 7, or about 5.5 to about 6.5. In at least one embodiment, the pH of the hair care composition is about 2.5 to about 6.5 or about 3 to about 5.5.

In certain embodiments, the hair care composition has a viscosity of from about 1 to about 50,000 centipoise (cP), about 5 to about 40,000 cP, or about 10 to about 20,000 cP, or about 100 to about 10,000 cP, including all values in between these ranges, including all values in between these ranges, at room temperature using Brookfield viscometer with a Spindle E at a speed of 12 rpm.

Additional ingredients may be present in the hair care composition. These include water and ingredients to thicken, preserve, emulsify, add fragrance, adjust the pH, and color. The hair care compositions may include any of the following additional ingredients in an amount of from about 0.01 to about 5 wt. %. In some instances, the amount of additional ingredients present in the hair care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the hair care composition.

The hair care composition can comprise additional ingredients, such as proteins (e.g., hydrolyzed vegetable protein, hydrolyzed wheat protein, hydrolyzed milk protein, hydrolyzed silk and hydrolyzed collagen), vitamins (e.g., panthenol, biotin, vitamin E acetate, vitamin A and D palmitate), moisturizers/humectants (e.g., glycerin, propylene glycol, sodium pyroglutamic acid (also known as PCA), amino acid-based surfactants, and HLA), emollients (e.g., esters, isopropyl myristate, decyl oleate, C12-15 alkyl benzoate), botanicals (e.g., chamomile, aloe, rosemary), as well as preservatives, dyes, pH adjusters and chelating agents. Additional examples of vitamins that may be included in some hair care compositions include tocopherol, retinol, and ascorbic acid. Vitamin derivatives, such as ascorbyl monopalmitate, tocopheryl acetate, and Vitamin E palmitate, may also be included in certain embodiments of the invention. Any one of these additional ingredients may be present in the compositions of the present invention in an amount of from about 0.001 wt. % to about 5 wt. %., from about 0.01 wt. % to about 2.5 wt. %, or from about 0.1 wt. % to about 1 wt. %, based on the total weight of the composition.

Non-limiting examples of fragrances and perfumes include odor compounds selected from: 7-acetyl-1,2,3,4,5,6,7,8-octahydro-1,1,6,7-tetramethylnaphthalene, α-ionone, β-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl) propionaldehyde, ethylvanillin, heliotropin, hexylcinnamaldehyde, amylcinnamaldehyde, 2-methyl-2-(isopropylphenyl) propionaldehyde, coumarin, γ-decalactone, cyclopentadecanolide, 16-hydroxy-9-hexadecenoic acid lactone, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, and tert-butylcyclohexyl acetate.

Other fragrances may include odor compounds selected from essential oils, resinoids and resins from a large number of sources, such as, for example, Peru balsam, olibanum resinoid, styrax, labdanum resin, nutmeg, cassia oil, benzoin resin, coriander, and lavandin.

Further suitable fragrances include odor compounds selected from phenylethyl alcohol, terpineol, linalool, linalyl acetate, geraniol, nerol, 2-(1,1-dimethylethyl)cyclo-hexanol acetate, benzyl acetate, and eugenol. The fragrances or perfumes can be used as single substances or in a mixture with one another.

The hair care composition may further comprise one or more colorants. The colorants may be a pigment, a dye, or mixtures thereof. Non-limiting examples of pigments include titanium dioxide, Zinc Oxide, Kaolin, Mica etc. Non-limiting examples of dyes include food dyes suitable for food, drug and cosmetic applications, and mixtures thereof. Some color agents (colorants) are known as FD&C dyes.

The colorants may be present in an amount ranging from about 0.0001% wt. % to about 0.4% wt. %, including all percentages and subranges therebetween, based on the total weight of the hair care composition. In some embodiments, the colorants may be present in an amount ranging from about 0.0001% wt. % to about 4% wt. %, including all percentages and subranges therebetween, based on the total weight of the hair care composition.

In certain embodiments, in order to prevent ingredients from separating, emulsifiers may be added. In certain embodiments, additional ingredients may include conditioning agents that moisturize the skin during and after use of the hair care composition.

In other embodiments, the hair care composition may include any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents; sequestrants, for example, tetrasodium EDTA, and/or their equivalents; organic acids, for example, citric acid and/or formic acid and/or their equivalents; viscosity modifiers; fragrances and/or perfumes; preservatives, for example, phenoxyethanol, formaldehyde solution, parabens, pentanediol or sorbic acid; and dyes and pigments that are approved and suitable for cosmetic purposes.

In some embodiments, the compositions of the present invention may include water in an amount from about 1 to about 95 wt. %, based on the total weight of the hair care composition. For example, the water may be present in the hair care composition in an amount from about 5 to about 95 wt. %, about 10 to about 95 wt. %, about 15 to about 95 wt. %, about 20 to about 95 wt. %, about 30 to about 95 wt. %, about 40 to about 95 wt. %, about 50 to about 95 wt. %, about 60 to about 95 wt. %, about 70 to about 95 wt. %, about 80 to about 95 wt. %, about 90 to about 95 wt. %; about 5 to about 90 wt. %, about 10 to about 90 wt. %, about 15 to about 90 wt. %, about 20 to about 90 wt. %, about 30 to about 90 wt. %, about 40 to about 90 wt. %, about 50 to about 90 wt. %, about 60 to about 90 wt. %, about 70 to about 90 wt. %, about 80 to about 90 wt. %; about 5 to about 80 wt. %, about 10 to about 80 wt. %, about 15 to about 80 wt. %, about 20 to about 80 wt. %, about 30 to about 80 wt. %, about 40 to about 80 wt. %, about 50 to about 80 wt. %, about 60 to about 80 wt. %, about 70 to about 80 wt. %; about 5 to about 70 wt. %, about 10 to about 70 wt. %, about 15 to about 70 wt. %, about 20 to about 70 wt. %, about 30 to about 70 wt. %, about 40 to about 70 wt. %, about 50 to about 70 wt. %, about 60 to about 70 wt. %.

Some embodiments of the present invention provide a composition for promoting the growth and/or reducing the loss of hair in a mammalian subject, the composition comprising: from about 60 wt % to about 70 wt. % (e.g., about 60 wt. %, about 61 wt. %, about 62 wt. %, about 63 wt. %, about 64 wt. %, about 65 wt. %, about 66 wt. %, about 67 wt. %, about 68 wt. %, about 69 wt. %, or about 70 wt. %) water; from about 5 wt. % to about 15 wt. % (e.g., about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, or about 15 wt. %) ethanol; from about 5 wt. % to about 10 wt. % (e.g., about 5 wt. %, about 6 wt. %, about 7 wt. %, about 8 wt. %, about 9 wt. %, or about 10 wt. %) butylene glycol; from about 1 wt. % to about 5 wt. % (e.g., about 1 wt. %, about 2 wt. %, about 3 wt. %, about 4 wt. %, or about 5 wt. %) Alanine/Histidine/Lysine Polypeptide Copper HCl; from about 1 wt. % to about 4 wt. % (e.g., about 1 wt. %, about 2 wt. %, about 3 wt. %, or about 4 wt. %) betaine; from about 0.5 wt. % to about 2 wt. % (e.g., about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. %) hyaluronic acid, or a salt thereof; from about 0.5 wt. % to about 2 wt. % (e.g., about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. %) of a complex comprising: Larix Europaca Wood Extract; Camellia Sinensis Leaf Extract and a zinc ion source; from about 0.5 wt. % to about 2 wt. % (e.g., about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. %) of a complex comprising: mannitol; caffeine; a zinc ion source; Aesculus Hippocastanum (Horse Chestnut) Seed Extract; and glycyrrhizic acid, or a salt thereof; from about 0.5 wt. % to about 2 wt. % (e.g., about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. %) Serenoa Serrulata Fruit Extract; and from about 0.5 wt. % to about 2 wt. % (e.g., about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. %) Eriobotrya Japonica Leaf Extract; and from about 0.05 wt. % to about 0.25 wt. % (e.g., about 0.05 wt. %, about 0.06 wt. %, about 0.07 wt. %, about 0.08 wt. %, about 0.0 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.20 wt. %, about 0.21 wt. %, about 0.22 wt. %, about 0.23 wt. %, about 0.24 wt. %, or about 0.25 wt. %) hexamidine diisethionate.

In some embodiments, the compositions of the present invention further comprise from about 0.1 wt. % to about 1 wt % of one or more of the following ingredients: Phellinus Linteus Extract; Glycyrrhiza Uralensis (Licorice) Root Extract; Houttuynia Cordata Extract; Vitis Vinifera (Grape) Seed Extract; Aronia Arbutifolia Extract; Malus Domestica Fruit Extract; Wine Extract; Perilla Frutescens Leaf Extract; Camellia Sinensis Leaf Extract; Palmitoyl Tripeptide-1; and Copper Tripeptide-1. In some embodiments, the compositions comprise about 0.9 wt. %, about 0.8 wt. %, about 0.7 wt. %, about 0.6 wt. %, about 0.5 wt. %, about 0.4 wt. %, about 0.3 wt. %, about 0.2 wt. %, or about 0.1 wt. %, of one or more of the following ingredients: Phellinus Linteus Extract; Glycyrrhiza Uralensis (Licorice) Root Extract; Houttuynia Cordata Extract; Vitis Vinifera (Grape) Seed Extract; Aronia Arbutifolia Extract; Malus Domestica Fruit Extract; Wine Extract; Perilla Frutescens Leaf Extract; Camellia Sinensis Leaf Extract; Palmitoyl Tripeptide-1; and Copper Tripeptide-1.

In some embodiments, compositions of the present invention further comprise from about 0.01 wt. % to about 0.1 wt. % of one or more of the following ingredients: Aloe Barbadensis Leaf Extract; Saccharomyces/Copper Ferment; Saccharomyces/Zinc Ferment; apigenin; oleanoilic acid; and Mentha Piperita (Peppermint) Oil. In some embodiments, compositions of the present invention further comprise about 0.09 wt. %, about 0.08 wt. %, about 0.07 wt. %, 0.06 wt. %, 0.05 wt. %, about 0.04 wt. %, about 0.03 wt. %, about 0.02 wt. %, about 0.015 wt. %, or about 0.01 wt. %, of one or more of the following ingredients: Aloe Barbadensis Leaf Extract; Saccharomyces/Copper Ferment; Saccharomyces/Zinc Ferment; apigenin; oleanoilic acid; and Mentha Piperita (Peppermint) Oil.

In other embodiments, the compositions of the present invention further comprise from about 0.5 wt. % to about 2 wt. %, of one or more of the following ingredients: a complex comprising: glycerin, sorbitol and Pterocarpus Marsupium Bark Extract; and soy isoflavones. In further embodiments, the compositions of the present invention further comprise from about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. % or about 2 wt. %, of one or more of the following ingredients: a complex comprising: glycerin, sorbitol and Pterocarpus Marsupium Bark Extract; and soy isoflavones.

### EXAMPLES

### Example 1

A more detailed description of an exemplary composition of the present invention is provided below in Table 1. As shown therein, the functionality of various ingredients is provided.

TABLE 1

| Ingredient | Mechanism |
|---|---|
| Larix europaea wood extract (taxifolin, | potent anti-inflammatory |
| DHQG) | |
| Zinc chloride | inhibits Malassezia furfur |
| Camellia sinensis Leaf Extract | 5α-reductase inhibitor |
| (epigallocatechin gallate, EGCG) | |
| Alanine/Histidine/Lysine Polypeptide | peptides complex, signals for cell rejuvenation in |
| Copper HCl | the dermis, i.e., restructuring the dermis |
| | including hair follicles |
| Betaine | detoxifies homocysteine |
| Caffeine | phosphodiesterase inhibitor, leading to vascular |
| | dilation |
| Aesculus hippocastanum (Horse | anti-inflammatory and vascularization |
| Chestnut) seed extract | |
| Sodium hyaluronate | rebuilds the subdermal extra cellular matrix |
| Eriobotrya japonica (Loquat) leaf | Shown to Stimulate FGF-7, VEGF, BMP-2 which |
| extract | promotes hair growth and suppress FGF-5, |
| | which induces hair loss and increases blood |
| | circulation |
| Glycyrrhiza uralensis (Licorice) root | prenylated isoflavones, potent antimicrobial |
| extract | |
| Phellinus linteus (mushroom) extract | β-glucan and terpenes, anti-inflammatory, |
| | promotes keratinocyte growth and proliferation |
| Houttuynia cordata extract | traditional medicine for hair, possible mechanism |
| | is local glucose modulation |
| Vitis vinifera (Grape) seed extract | procyanidins such as B2 and B3, promote anagen |
| | hair growth by reducing PKC isozymes and |
| | TGF-beta signaling |
| Aronia arbutifolia Extract | rich in antioxidant polyphenols |
| Malus domestica (apple) fruit extract | procyanidin B2 reduces PKC isozymes that |
| | signal for telogen |
| Wine extract | flavan-3-ols, potent antioxidants |
| Perilla frutescens leaf extract | rosmarinic acid, antioxidant, 5α-reductase |
| | inhibitor |
| Apigenin | anti-inflammatory |
| Oleanolic Acid | anti-inflammatory |
| Palmitoyl tripeptide-1 | GHK with fatty acid ester, fat soluble peptide for |
| | rejuvenation |
| Biotinoyl tripeptide-1 | GHK with biotin ester, bioavailable peptide for |
| | rejuvenation |
| Copper peptide-1 | Cu-GHK, signals for rejuvenation |
| Glycogen | hair follicle food source |
| Aloe barbadensis leaf extract | anti-inflammatory oligosaccharide |
| Saccharomyces/Copper Ferment, | bioavailable copper and zinc |
| Saccharomyces/Zinc Ferment | |
| Mentha piperita (Peppermint) Oil, and | activates cold menthol receptor, increases |
| Menthol | penetration of ingredients |
| Houttuynia cordata extract | rich in antioxidant, anti-inflammatory flavonols |
| Hexamidine diisethionate | show to have potent antimicrobial efficacy |
| Aronia arbutifolia extract | the natural oil moisturizes and conditions hair |

An exemplary composition tailored for a male subject is described below in Table 2.

TABLE 2
Ex. I

| Ingredients | | Wt. % |
|---|---|---|
| | Water | 60-70 |
| | Alcohol | 1-15 |
| | Butylene Glycol | 1-15 |
| | Alanine/Histidine/Lysine Polypeptide | 0.1-5 |
| | Copper HCl | |
| | Betaine | 0.1-5 |
| | Sodium Hyaluronate | 0.1-3 |
| Redensyl | Glycerin | 0.1-3 |
| | Water | |
| | Larix Europaea Wood Extract | |
| | Glycine | |
| | Camellia Sinensis Leaf Extract | |
| | Zinc Chloride | |
| | Sodium Metabisulfite | |
| Anasensyl | Mannitol | 0.1-3 |
| | Ammonium Glycyrrhizate | |
| | Caffeine | |
| | Zinc Gluconate | |
| | Aesculus Hippocastanum (Horse Chestnut) | |
| | Seed Extract | |
| Saw Palmetto | Water | 0.1-3 |
| Extract | Butylene Glycol | |
| | Serenoa Serrulata Fruit Extract | |
| | Phenoxyethanol | |
| Loquat Leaf | Butylene Glycol | 0.1-3 |
| Extract CA | Eriobotrya Japonica Leaf Extract | |
| | PEG-40 Hydrogenated Castor Oil | 0.1-2 |
| | Phellinus Linteus Extract | 0.1-2 |
| | Glycyrrhiza Uralensis (Licorice) Root Extract | 0.1-2 |
| | Houttuynia Cordata Extract | 0.1-2 |
| | Vitis Vinifera (Grape) Seed Extract | 0.1-2 |
| | Aronia Arbutifolia Extract | 0.1-2 |
| | Malus Domestica Fruit Extract | 0.1-2 |
| | Wine Extract | 0.1-2 |
| | Perilla Frutescens Leaf Extract | 0.1-2 |
| | Camellia Sinensis Leaf Extract | 0.1-2 |
| | Biosaccharide Gum-1 | 0.1-2 |
| | Caprylhydroxamic Acid | 0.1-2 |
| | Caprylyl Glycol | |
| | Glycerin | |
| | Glyceryl Polymethacrylate | 0.1-2 |
| | Propylene Glycol | |
| | Palmitoyl Tripeptide-1 | |
| | Phenoxyethanol | 0.1-2 |
| Elestap | Hexamidine diisethionate | 0.05-0.25 |
| | Phanthenol | 0.05-0.25 |
| Copper | Copper Tripeptide-1 | 0.05-0.25 |
| Tripeptide-1 | | |
| | Glycerin | 0.05-0.25 |
| | Glyceryl Acrylate/Acrylic Acid Copolymer | |
| | Propylene Glycol | |
| | Butylene Glycol | 0.05-0.25 |
| | Water | |
| | PPG-26-Buteth-26 | |
| | PEG-40 Hydrogenated Castor Oil | |
| | Apigenin | |
| | Oleanolic Acid | |
| | Biotinoyl Tripeptide-1 | |
| | Glycogen | 0.01-0.1 |
| | Aloe Barbadensis Leaf Extract | 0.01-0.1 |
| | Flavor | 0.01-0.1 |
| | Disodium EDTA | 0.01-0.1 |
| | Saccharomyces/Copper Ferment | 0.01-0.1 |
| | Saccharomyces/Zinc Ferment | 0.01-0.1 |
| | Sodium Citrate | 0.01-0.1 |
| | Mentha Piperita (Peppermint) Oil | 0.005-0.025 |
| | Lactic Acid | 0.00100 |
| | Menthol | 0.00100 |

| | |
|---|---|
| Total | 100.00000 |

An exemplary composition tailored for a female subject is described below in Table 3.

TABLE 3
Ex. II

| Ingredients | | Wt. % |
|---|---|---|
| | Water | 60-70 |
| | Alcohol | 1-15 |
| | Butylene Glycol | 1-15 |
| | Alanine/Histidine/Lysine Polypeptide | 0.1-5 |
| | Copper HCl | |
| | Betaine | 0.1-5 |
| | Sodium Hyaluronate | 0.1-3 |
| Redensyl | Glycerin | 0.1-3 |
| | Water | |
| | Larix Europaea Wood Extract | |
| | Glycine | |
| | Camellia Sinensis Leaf Extract | |
| | Zinc Chloride | |
| | Sodium Metabisulfite | |
| Anasensyl | Mannitol | 0.1-3 |
| | Ammonium Glycyrrhizate | |
| | Caffeine | |
| | Zinc Gluconate | |
| | Aesculus Hippocastanum (Horse Chestnut) | |
| | Seed Extract | |
| Tricholastyl | Glycerin | 0.1-3 |
| | Sorbitol | |
| | Pterocarpus Marsupium Bark Extract | |
| | Glutamic Acid | |
| | Disodium Succinate | |
| | Water | |
| Soy | Water | 0.1-3 |
| Isoflavones | Butylene Glycol | |
| | Soy Isoflavones | |
| | Phenoxyethanol | |
| Loquat Leaf | Butylene Glycol | 0.1-3 |
| Extract CA | Eriobotrya Japonica Leaf Extract | |
| | PEG-40 Hydrogenated Castor Oil | 0.1-2 |
| | Phellinus Linteus Extract | 0.1-2 |
| | Glycyrrhiza Uralensis (Licorice) Root Extract | 0.1-2 |
| | Houttuynia Cordata Extract | 0.1-2 |
| | Vitis Vinifera (Grape) Seed Extract | 0.1-2 |
| | Aronia Arbutifolia Extract | 0.1-2 |
| | Malus Domestica Fruit Extract | 0.1-2 |
| | Wine Extract | 0.1-2 |
| | Perilla Frutescens Leaf Extract | 0.1-2 |
| | Camellia Sinensis Leaf Extract | 0.1-2 |
| | Biosaccharide Gum-1 | 0.1-2 |
| | Caprylhydroxamic Acid | 0.1-2 |
| | Caprylyl Glycol | |
| | Glycerin | |
| | Glyceryl Polymethacrylate | 0.1-2 |
| | Propylene Glycol | |
| | Palmitoyl Tripeptide-1 | |
| | Phenoxyethanol | 0.1-2 |
| Elestap | Hexamidine diisethionate | 0.05-0.25 |
| | Phanthenol | 0.05-0.25 |
| Copper | Copper Tripeptide-1 | 0.05-0.25 |
| Tripeptide-1 | | |
| | Glycerin | 0.05-0.25 |
| | Glyceryl Acrylate/Acrylic Acid Copolymer | |
| | Propylene Glycol | |
| | Butylene Glycol | 0.05-0.25 |
| | Water | |
| | PPG-26-Buteth-26 | |
| | PEG-40 Hydrogenated Castor Oil | |
| | Apigenin | |
| | Oleanolic Acid | |
| | Biotinoyl Tripeptide-1 | |
| | Aloe Barbadensis Leaf Extract | 0.01-0.1 |
| | Flavor | 0.01-0.1 |
| | Disodium EDTA | 0.01-0.1 |
| | Saccharomyces/Copper Ferment | 0.01-0.1 |
| | Saccharomyces/Zinc Ferment | 0.01-0.1 |
| | Sodium Citrate | 0.01-0.1 |
| | Mentha Piperita (Peppermint) Oil | 0.005-0.025 |
| | Lactic Acid | 0.00100 |
| | Menthol | 0.00100 |

| | |
|---|---|
| Total | 100.00000 |

The compositions described in Tables 2 and 3 can be prepared by conventional means known to those skilled in the art.

### Example 2

An observational four-month study to measure the efficacy of an exemplary composition of the present invention for hair regrowth in participants with androgenetic alopecia was performed. Five participants applied the topical roller once or twice daily to the areas of their scalp where hair loss was evident. Participants were assessed at study days 0, 60, and 120. A patient satisfaction questionnaire was filled out at the end of the study and photography was taken at days 0 and 120. Digital photographs were taken with a standardized digital camera utilizing consistent settings, lighting, and standardized positioning. The Capily Gro-Trac hair analysis system (Capily Institute, Los Angeles CA) was used for hair counts. This device and software allow for automated measurements and calculations of both vellus and terminal hairs per unit area. The same area was photographed and analyzed at each time point.

The primary objective of this study was to measure the change in hair density using automated image analysis with the Capily Gro-Trac system. A secondary objective was to assess the satisfaction of participants utilizing a questionnaire.

### Inclusion Criteria

1. Males and Females between 21-60 years of age, inclusive
2. Have self-reported thinning or hair loss for more than six (6) months before screening
3. Clinically confirmed to have hair loss or thinning by the investigator via physical exam.
4. In good general health, as determined by the Investigator
5. Willing and able to attend all study visits
6. Willing to maintain the same hairstyle as at the Screening Visit for the duration of the study

### Exclusion Criteria

1. Scalp hair loss on the treatment area, due to disease, injury, or medical therapy
3. Subjects who are pregnant, planning to become pregnant, or breastfeeding.
4. Current skin disease (e.g., psoriasis, atopic dermatitis, skin cancer, eczema, sun damage, seborrheic dermatitis), cuts and or abrasions on the scalp or condition (e.g., sunburn) on the treatment area that, in the opinion of the Investigator, might put the subject at risk or interfere with the study conduct or evaluations.
5. No history of burning, flaking, itching, and stinging of the scalp.

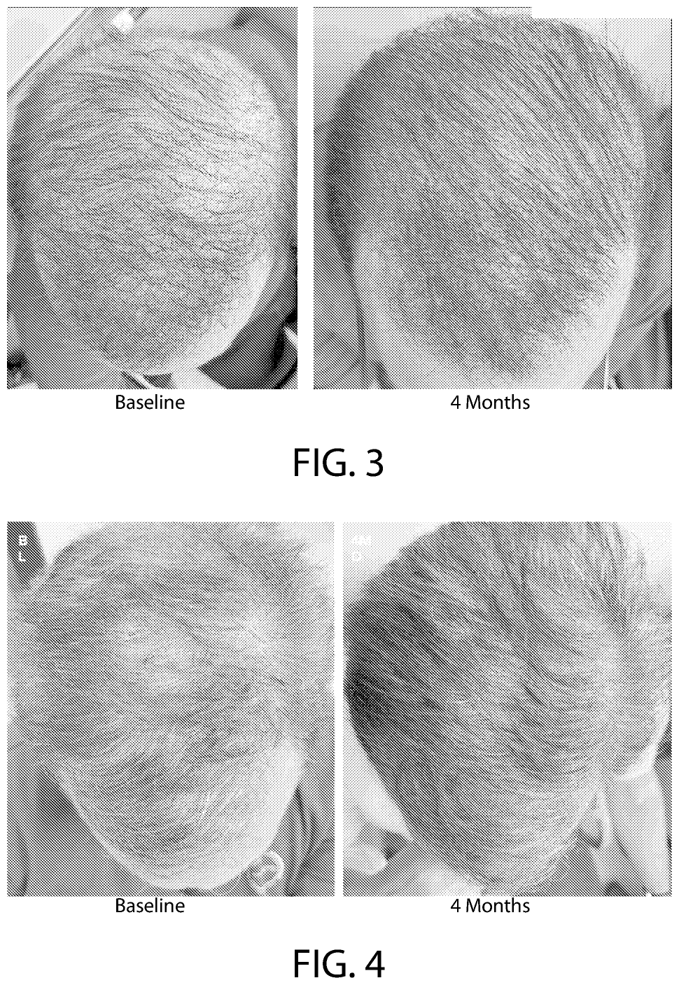

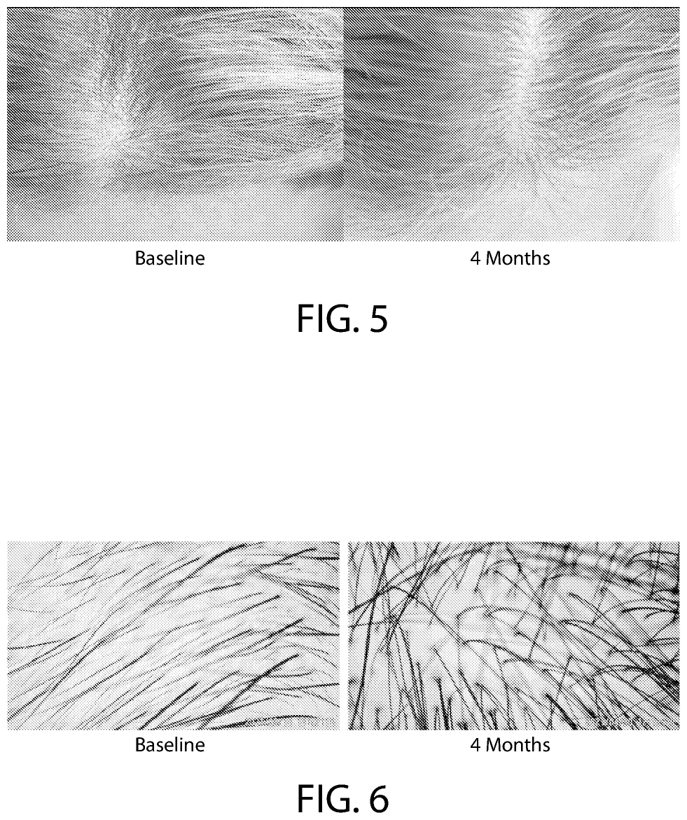

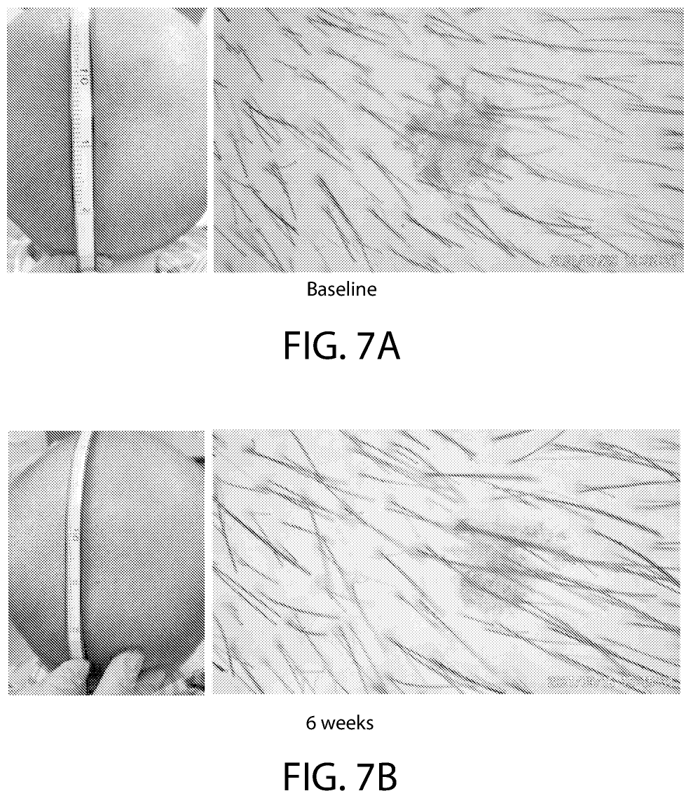

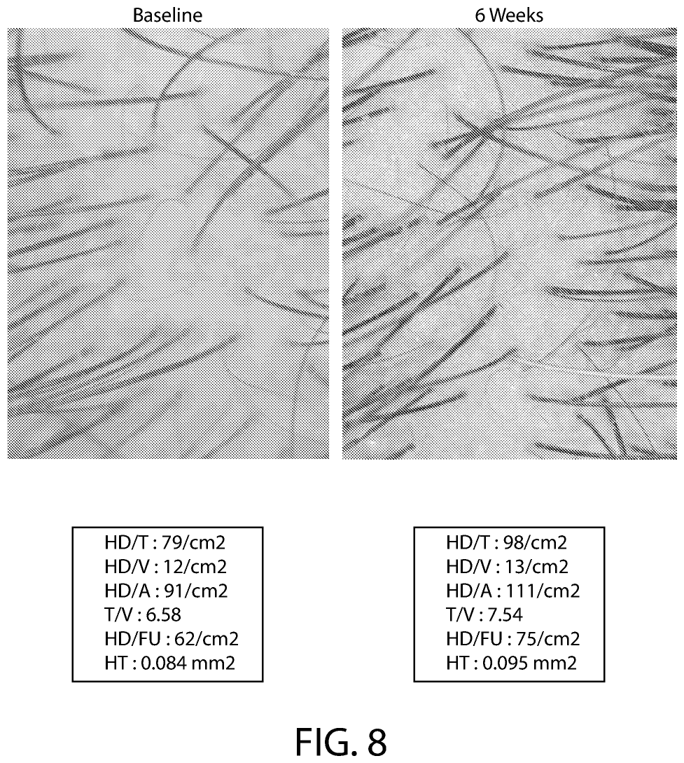

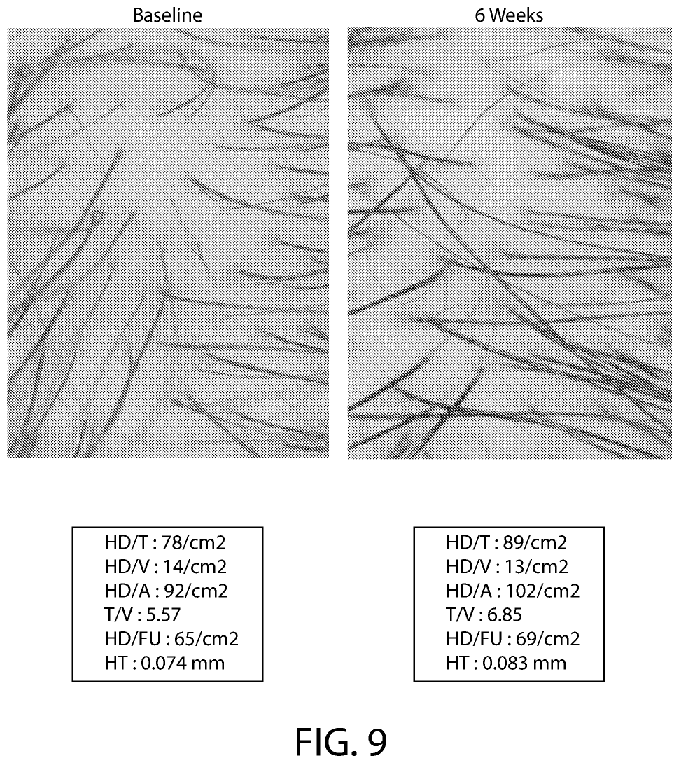

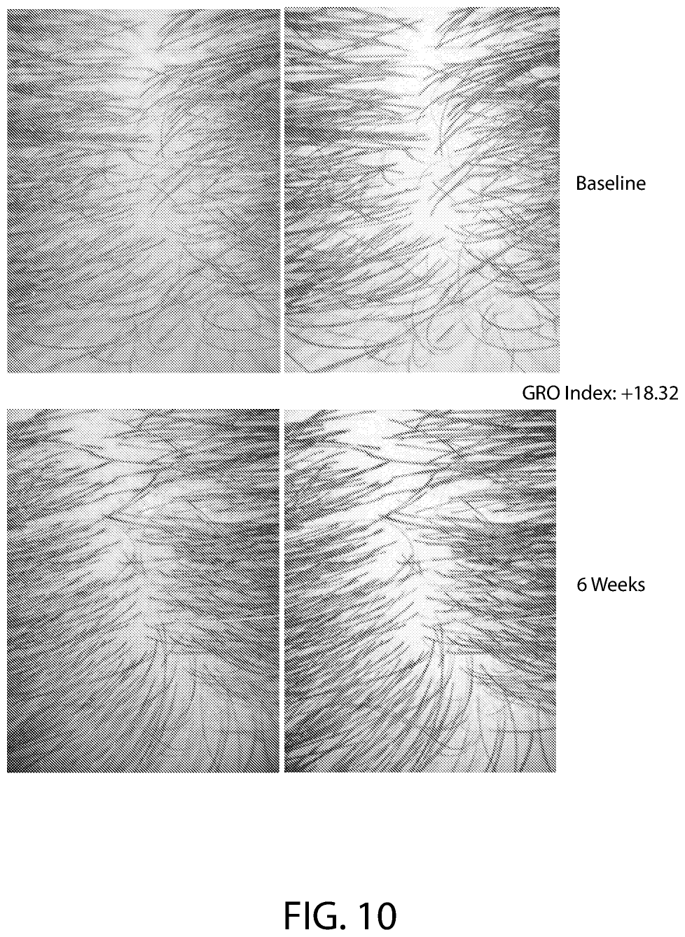

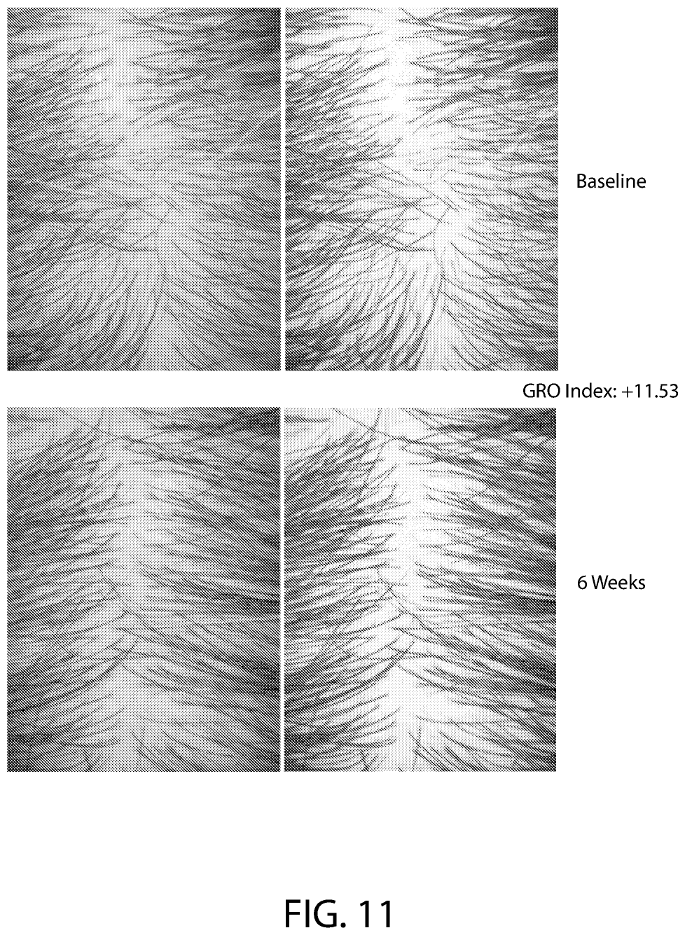

A total of 5 androgenetic alopecia participants were included in the study with all 5 participants completing the study. At the end of the study, the density per square centimeter was statistically significantly greater from before to after treatment. Additionally, the median terminal density per sq cm significantly increased in comparison to measurements taken before therapy. Furthermore, the median vellus density per sq cm was also significantly increased following therapy with an exemplary composition of the present invention. Global and trichoscopic images pre- and post-four months of use of an exemplary composition of the present invention can be seen in FIGS. 3-6.

Our results support the use of the hair care compositions described herein to treat AGA. Additionally, the compositions of the present invention are naturally derived (drug-free) and were well-tolerated in our study.

Overall, there are numerous options when it comes to treating AGA, including over-the-counter and prescription medications, in addition to nutraceuticals. The compositions of the present invention differ from others on the market in the ingredients target various causes of hair loss, and the formula is specific to men and women. This study supports the use of compositions of the present invention as monotherapy to treat AGA. We found that the median total hair density per square centimeter, median terminal density per square centimeter, and median vellus density per square centimeter were all significantly increased after treatment for 120 days.

This study supports future studies to further quantify the effects on various types of hair loss.

### Example 3

An open-label, prospective, observational study was conducted. The study was conducted according to the Declaration of Helsinki and the ICH GCP guidelines as applicable to cosmetic products. All subjects received the patient information and provided written, informed consent, including photography.

Enrolled subjects were male and female adults aged 18 to 75 years of age with a diagnosis of androgenetic alopecia (AGA). Clinical severity ranged from mild to severe. Subjects with evidence of hair loss for reasons other than AGA were excluded. Use of oral or topical hair growth products was not permitted in the 4 weeks preceding the study and during the study. During the study, participants were instructed to not use any hair treatments that could affect hair loss, and not to change their usual hair hygiene habits, diet, exercise routine, or contraceptive method. Anti-inflammatories, antihistamines, immunosuppressive and retinoids were prohibited during the study.

Participants applied an exemplary composition (Ex. I or Ex. II) of the present invention at home, in accordance with the product's instructions for use: the roller was massaged in circular motions all over the frontal, crown and vertex areas of the scalp. The product was applied twice per day, and left on overnight.

Adverse events were monitored throughout the study. A dermatologist assessed for erythema, edema, vesicles, papules, macules, crust, dryness, and dyschromia, and participants were questioned about any feelings of discomfort, such as burning or stinging at followup (8 weeks after baseline). Efficacy assessments included digital phototrichogram for a subset of patients, digital photography, and patient questionnaires. For the phototrichogram assessment, images were taken using the computerized trichogram Tricho Scan (Dermoscan GmbH, Regensburg, Germany) and changes in hair thickness and density were calculated at week 8 from baseline. Statistical analysis was performed with a paired t-test to compare before and after data for each parameter measured on trichogram. Patient questionnaires were completed 8 weeks after treatment initiation. Participants assessed the product's effects and cosmetic qualities via a 5-point likert scale (greatly improved, slightly improved, no change, slightly worsened, greatly worsened), and yes/no questions on satisfaction: whether its easy to use, whether it affects the hair styling and whether they will continue using the product.

150 subjects were enrolled in the study and compliance was 100%. Baseline characteristics of patients are shown in Table 4 (below): mean age was 56.4 years, and 60% of participants were female.

TABLE 4
Parameter

| | |
|---|---|
| Average age (range) years | 56.4 (38-75) |
| Female % | 60% |
| Skin Type | |
| I | 10% |
| II | 20% |
| III | 40% |
| IV | 10% |
| V | 15% |
| VI | 5% |

None of the participants reported discomfort and no adverse reactions were detected on examination.

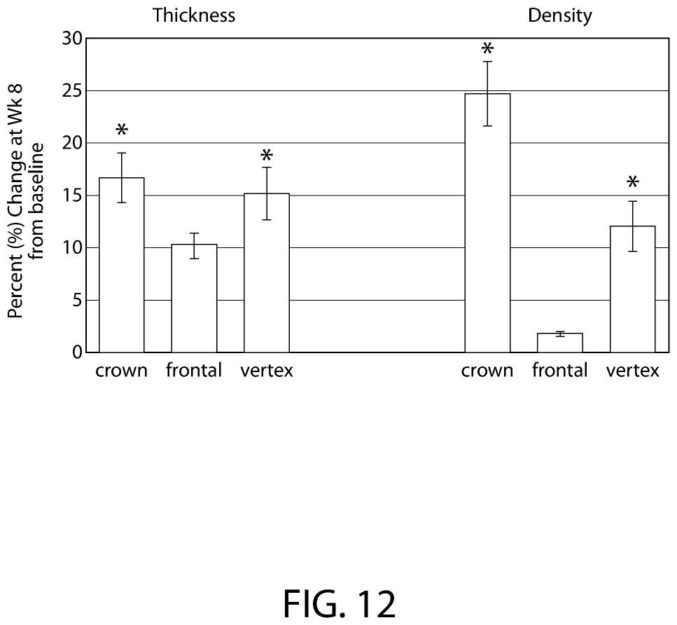

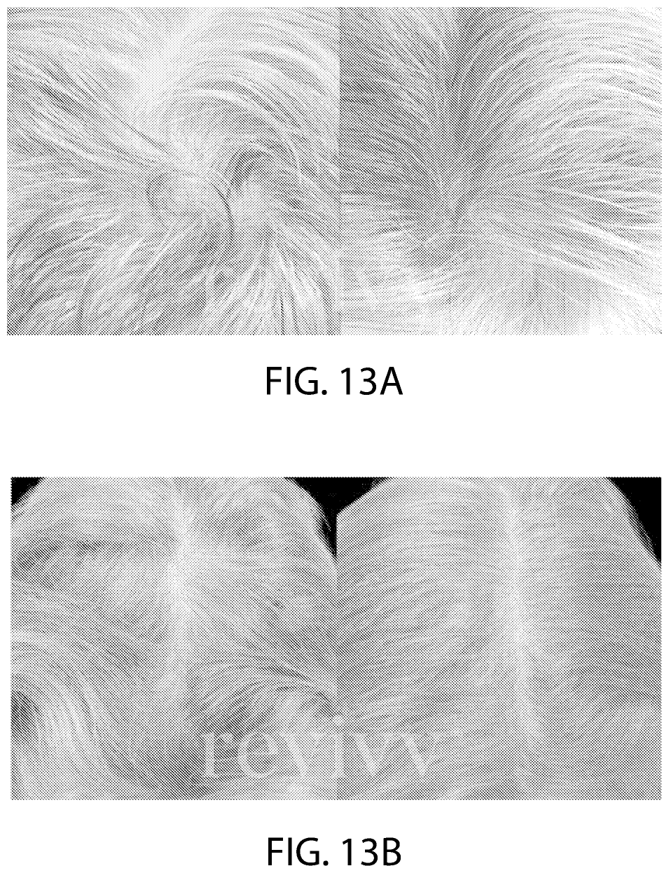

Seven patients participated in the phototrichogram substudy. Phototrichogram analysis showed a statistically significant increase in hair thickness in two areas (crown, vertex) at week 8 from baseline (see, FIG. 12). Representative images from a male and female patient with AGA after 8 weeks of treatment are shown in FIG. 13.

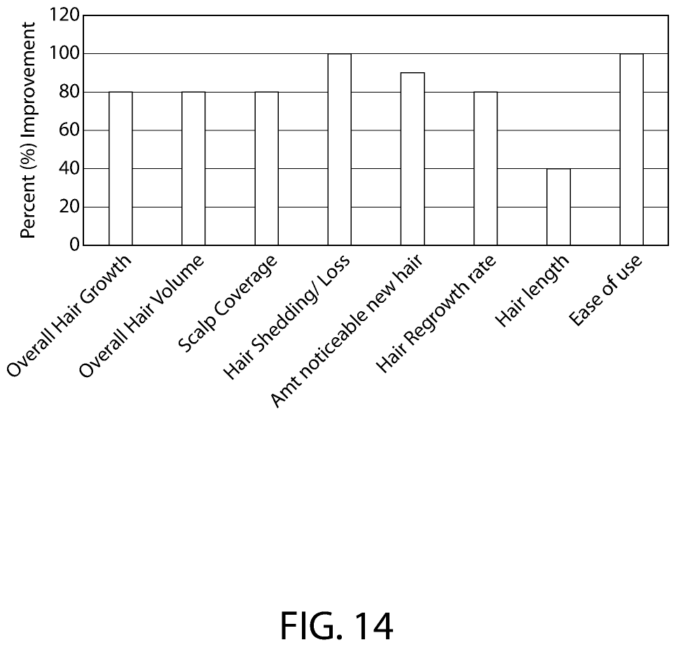

In a participant questionnaire, the product was rated highly both for efficacy and cosmetic qualities. At week 8, 80% of participants noted increase in hair growth, hair volume, scalp coverage, and amount of hair regrowth. Ninety percent of participants noticed increased amount of new hair while all participants said the hair shedding decreased and the product was easy to use (FIG. 14). Cosmetically, the product was rated very highly: all patients said the product was easy to use, did not affect styling, and most patients would continue to use it.

An exemplary composition of the present invention is shown in this study to improve hair growth in clinical and subjective efficacy parameters in 150 participants from a real-world clinical setting. Analysis of phototricograms at week 8 post twice daily use of the topic serum revealed significant increase in hair thickness and hair density in the vertex and crown areas of the scalp. Moreover, almost all participants found the product to be effective, easy to use and incorporate with their hair routine, and would continue using it.

Aside from efficacy, the cosmetic properties of topical hair growth serums should not negatively alter existing hair, and should be conducive with a variety of hair care routines in order to promote regular use. The results of this study, that was conducted in ethnically diverse participants, showed high level of subject satisfaction with both the hair growth benefits and the cosmetic properties. There were no reports of skin irritation, or dermatitis that sometime occurs with the use of botanicals.

As demonstrated by the data described in the foregoing Examples, the compositions and methods of the present invention provide clinically significant and visible hair growth—including, but not limited to, overall hair volume and scalp coverage—and in both men and women. The compositions and methods of the present invention were also able to significantly reduce hair loss.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. Further, it will be apparent to those of skill in the art that numerous changes may be made in such details without departing from the spirit and the principles of the invention.

1-46. (canceled)

47. A hair care composition comprising:
an androgen balancing agent;
a prostaglandin balancing agent;
an antimicrobial agent;
an anti-inflammatory agent;
a collagen modulator;
a circulation enhancer;
an amino acid; and
a cosmetically acceptable carrier.

48. The hair care composition according to claim 47, wherein the androgen balancing agent is selected from an extract or derivative of: Ganoderma lucidum (Reishi); Glycyrrhiza glabra (Licorice); Paeonia lactiflora (White Peony); Camellia sinensis (Green Tea); Mentha spicata (Spearmint); Actaea racemose (Black Cohosh); Vitex agnus-castus (Chaste Tree); Serenoa repens (Saw Palmetto); and a combination of two or more thereof.

49. The hair care composition according to claim 47, wherein the antimicrobial comprises an antibacterial agent or an antifungal agent.

50. The hair care composition according to claim 47, wherein the antimicrobial agent is selected from: a zinc ion source (e.g., zinc chloride or zinc gluconate); Glycyrrhiza uralensis (Licorice) root extract; hexamidine diisethionate; Saccharomyces/copper ferment; Saccharomyces/zinc ferment; ketonconazole; and a combination of two or more thereof.

51. The hair care composition according to claim 47, wherein the anti-inflammatory agent is selected from: Larix europaea wood extract (e.g., taxifolin or DHQG); Aesculus hippocastanum (Horse Chestnut) seed extract; Phellinus linteus (mushroom) extract; apigenin; oleanolic acid; Aloe barbadensis leaf extract; Houttuynia cordata extract; and a combination of two or more thereof.

52. The hair care composition according to claim 47, wherein the collagen modulator comprises hyaluronic acid, or a salt thereof.

53. The hair care composition according to claim 47, further comprising a penetration enhancer; wherein the penetration enhancer is selected from: peppermint oil; isopropyl myristate; a ceramide; lecithin; a liposome; and a combination of two or more thereof.

54. The hair care composition according to claim 47, wherein the hair care composition is selected from: a lotion; a serum; a tincture; a cream; an ointment; a gel; a powder; and a balm.

55. The hair care composition according to claim 47, wherein the circulation enhancer comprises a vasodilator; wherein the vasodilator is selected from: caffeine; cocoa; coenzyme Q10 (CoQ10); a source of Allium sativum; arginine; magnesium; niacin (vitamin B3); a nitrate source; Petroselinum crispum; Zingiber officinale; and a combination of two or more thereof.

56. The hair care composition according to claim 47, further comprising a peptide selected from: copper-tripeptie-1; glycyl-L-histidyl-L-lysine; palmitoyl tripeptide-1; biotinoyl tripeptide-1; and a combination of two or more thereof.

57. The hair care composition according to claim 47, wherein the amino acid is selected from: glutamic acid; methionine; cysteine; homocysteine; taurine; and a combination of two or more thereof.

58. The hair care composition according to claim 47, further comprising soy isoflavones.

59. The hair care composition according to claim 47, wherein the cosmetically acceptable carrier comprises:
from about 50 wt. % to about 80 wt. % water;
from about 5 wt. % to about 20 wt. % of a monohydric alcohol; and
from about 1 wt. % to about 15 wt. % of a diol.

60. A composition for promoting hair growth and/or reducing hair loss comprising:
a copper ion source;
an extract or derivative of Camellia sinensis;
an extract or derivative of Serenoa serrulate;
a natural vasodilator; and
a peptide selected from: copper-tripeptie-1; glycyl-L-histidyl-L-lysine; palmitoyl tripeptide-1; biotinoyl tripeptide-1; and a combination of two or more thereof.

61. A composition for promoting hair growth and/or reducing hair loss comprising:
a copper ion source;
an extract or derivative of Camellia sinensis;
soy isoflavones; and
a natural vasodilator.

62. A method for promoting hair growth and reducing hair loss comprising: administering an effective amount of a hair care composition according to claim 47, to a skin surface of a subject in need thereof.

63. The method according to claim 62, wherein the hair care composition is administered from a dispenser comprising an applicator; wherein the applicator comprises a perforated surface.

64. The method according to claim 62, wherein the hair care composition has a viscosity adapted to be delivered through said perforated surface of said applicator.

65. The method according to claim 62, wherein the administration of the hair care composition increases aggregation of intradermal adipocytes on the scalp of a mammalian subject; and wherein the increased aggregation of intradermal adipocytes results in a yellowing of the scalp.

66. The method according to claim 62, wherein the hair care composition increases the GRO Index by at least about 10.
